(19) United States
(12) Patent Application Publication
(10) Pub. No.: US 2007/0239104 A1
(43) Pub. Date: Oct. 11, 2007

(54) Anti acid G/tube pad

(76) Inventors: Angela C. Feerrar, Jersey Shore, PA (US); Tonja R. Pennycoff, Jersey Shore, PA (US)

Correspondence Address: WILLIAM J. DAVIS, ESQ., 11 CRESTVIEW LANE, SPARTA, NJ, 07871, US

(21) Appl. No.: 11/526,528

(22) Filed: Sep. 25, 2006

Related U.S. Application Data
(60) Provisional application No. 60/720,202, filed on Sep. 23, 2005.

(30) Foreign Application Priority Data
Sep. 26, 2005 (JP) 2005-278527
Dec. 7, 2005 (JP) 2005-353269
Mar. 2, 2006 (JP) 2006-055926

(51) Int. Cl. A61M 31/00

(52) U.S. Cl. 604/93.01

(57) ABSTRACT

An anti acid G/Tube pad comprising: a permeable layer adapted to face the skin, said permeable layer having thereon an antacid active, and an absorbent layer.

### CROSS REFERENCE TO RELATED APPLICATIONS

This application claims priority of U.S. provisional patent application No. 60/720,202 filed Sep. 23, 2005.

### BACKGROUND OF THE INVENTION

Gastrointestinal tubes are surgically implanted into the stomach through the abdominal wall for the purpose of the introduction of nutritional feeding. Many times the insertion of this tube becomes problematic due to the potential excretion of gastric acid through the incision site. Gastric acid is the normal content of the patient's stomach for the purpose of breaking down food. This gastric acid is very acidic and can become harmful when it comes in contact with the tissue outside of the stomach. In some cases, patients experience excoriation to the skin surrounding the GI tube site related to stomach acid secretions. This can lead to potential infection or damage to the site itself. It can be very detrimental to the patient's overall well-being.

Patients with gastrointestinal tubes many times experience altered/poor nutrition which leads to decreased healing of the body and results in exacerbation of the excoriated area.

Magnesium oxide is a neutralizing agent for gastric acid. It is usually given orally as a buffer or neutralizer to absorb hydrochloric acid in the stomach. A known intervention is to use a buffering agent which is magnesium oxide along with the active ingredient calcium carbonate. Applying the mixture of these agents externally to the abdominal tissue has proven to be effective in neutralizing hydrochloric acid.

### SUMMARY OF THE INVENTION

In accordance with this invention, there is provided a method to prevent or reduce stomach secretions from coming in contact with sensitive abdominal tissue and to prevent or reduce excoriation. The method comprises applying an anti acid G/tube pad of this invention as described below that adheres to the skin and enables good absorption of the acid/secretions.

### DETAILED DESCRIPTION OF THE ANTI ACID G/TUBE PAD OF THE INVENTION

The anti acid G/tube pad of the invention can be a single or multi-layered pad. In a preferred embodiment, it is a multi-layered pad with the outside layer being semi or non-permeable, but preferably semi-permeable. A non-permeable layer will contain the gastric acid within the pad, and a semi-permeable layer will contain the gastric acid but allow air to pass through. The pad can comprise an absorbing layer to absorb the gastric acid that is excreted from the G/tube site, and it can comprise a wicking layer to pull the gastric acid away from the G/tube area and into the absorbing layer. The absorbing layer preferably is between the outer layer and the wicking layer.

The antacid pad of the present invention preferably has both buffering and neutralizing properties. The buffering agent can reduce a patient's discomfort by functioning as a buffering agent to the skin surfaces preventing skin breakdown and excoriation.

The exposed surface of the inner layer of the anti acid G/tube pad features a region of an antacid active, for example, magnesium oxide and/or calcium carbonate and optionally an antacid free or discontinuous area. The antacid area will neutralize and prevent the breakdown of tissue around the G/tube related to stomach acid secretion. Suitable antacid materials are given in the Merck Index or the Code of Federal Regulations. Such antacids, other than magnesium hydroxide and calcium carbonate, are suggested for use in this invention. These are listed below:

Aluminum salts

Alexitol sodium (aluminum sodium carbonate hexitol complex)

Almagate (carbonic acid, aluminum magnesium complex)

aluminum hydroxide

aluminum magnesium silicate

aluminum phosphate

basic aluminum carbonate gel (aluminum hydroxide-aluminum carbonate gel)

Sucralfate (basic aluminum sucrose sulfate complex)

dihydroxyaluminum aminoacetate

dihydroxyaluminum sodium carbonate

Magaldrate (aluminum magnesium hydroxide monohydrate)

Bismuth salts

bismuth aluminate

bismuth phosphate

bismuth carbonate

bismuth subcarbonate

bismuth subgallate

bismuth subnitrate

Magnesium salts

magnesium carbonate

magnesium hydroxide

magnesium peroxide

magnesium phosphate, tribasic

magnesium silicates (magnesium trisilicate)

magnesium aluminosilicates

Other Salts of Bicarbonate, Citrate, Phospate, and Tartrate

sodium bicarbonate

potassium bicarbonate

potassium citrate

sodium potassium tartrate

tricalcium phosphate.

Any known buffering agent can be used in conjunction with the active antacid material.

In a preferred embodiment, the anti acid G/tube pad can contain approximately an average single adult dose of 1.3 grams of magnesium oxide and 1 gram of calcium carbonate. However, pads with much higher and lower amounts of antacid are contemplated within the scope of this invention.

The pad can comprise an antacid free area inside the pad, i.e., the skin facing surface area, that will be made up of 1 to 99, preferably 10 to 90, more preferably 20 to 80 and most preferably 30 to 50 percent of the total exposed skin facing surface area of the inner liner. By applying the antacid composition to the substrate in a discontinuous pattern, a portion of the skin-facing surface of the pad remains exposed to the skin to permit absorption of fluid such as gastric juices by the absorbent substrate.

The antacid can be disposed on the pad by a variety of techniques. For example, the antacid can be injected into the inner liner or it may be in the form of a coating that is placed on the exposed surface of the inner layer or in the form of a dispersed layer on the inner liner. The anti acid G/tube pad which holds an antacid, e.g., magnesium oxide and calcium carbonate, may have the medicament arranged in any suitable fashion. Many arrangements are contemplated, including a spiral arrangement.

The anti acid G/tube pad may have an adhesive edge on a portion of the outer edge of the inner layer so it may adhere to the patient's skin. The adhesive edge will create a seal and prevent leakage and protect the patient's tissue. The anti acid G/tube pad can have a non-permeable outer layer to prevent leakage as well. The pad can be square or circular in shape, preferably circular. However, other shapes are contemplated. In a preferred embodiment, the pad can have an opening or hole about the size of a G/tube approximately in the center of the pad. The pad preferably has a slit on the one side so it may be attached or removed without disrupting the tube.

Descriptions of the invention will be better understood by viewing the drawings below.

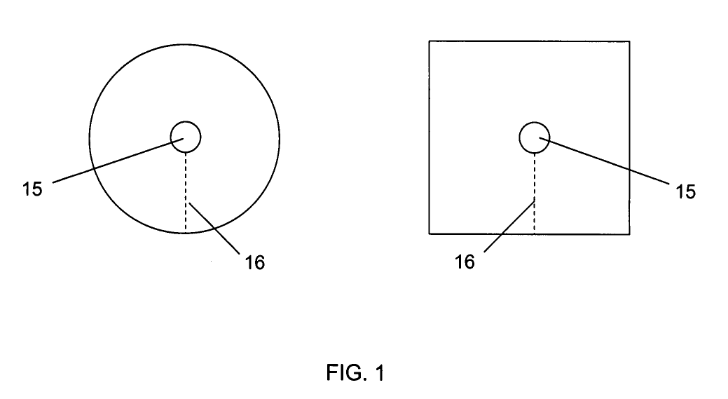

FIG. 1 is a rear view of the anti acid G/tube pad.

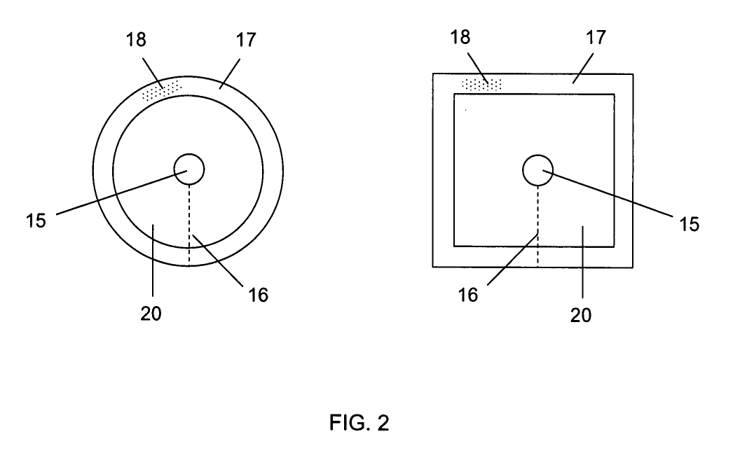

FIG. 2 is a front view of the anti acid G/tube pad.

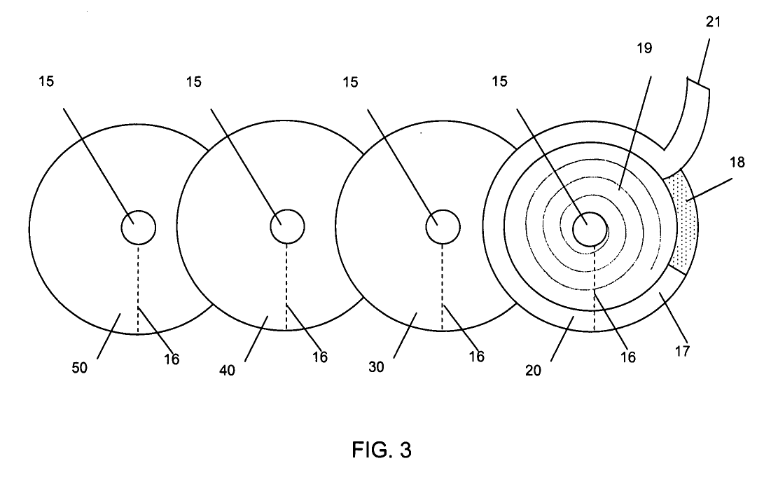

FIG. 3 is an exploded view of the layers of a preferred anti acid G/tube pad of the invention.

FIG. 1 depicts the pad of this invention as a flat circle or square shaped. It is an anti acid G/tube pad. In a preferred embodiment, it is to be made to fit around the G/tube and prevent the gastric acid/secretions from damaging or irritating the tissue surrounding the G/tube. The circular opening in the center 15 of the anti acid G/tube pad is where the G/tube can be positioned. The pad preferably can be held into place by any suitable adhesive as discussed below. The dotted line 16 can be perforated or a slit, preferably a slit. This is desirable so the anti acid G/tube pad may be attached or removed without disrupting the G/tube.

FIG. 2 is a front view of a preferred embodiment of the anti acid G/tube pad of this invention and shows inner liner 20. The inner layer or liner 20 can attach itself to the patient by the use of adhesive material 18 which is located on the outer edge or adhesive edge 17 of the inner liner 20. The adhesive 18 will ensure that the anti acid G/tube pad stays in position and self contains the patient's gastric acid within the anti acid G/tube pad. The adhesive edge 17 will create a seal that will prevent leakage and help to protect the patient's tissue. Any suitable adhesive known to be useful in similar medical applications can be used. If the adhesive is not present, the pad can be positioned in place by other techniques known in the art.

FIG. 3 is a view of the layers of a preferred anti acid G/tube pad.

The first layer or the outer layer 50 of the anti acid G/tube pad is a non permeable or a semi-permeable material, preferably a semi permeable material. Both materials will prevent gastric acid from passing through the anti acid G/tube pad, but a semi-permeable will also allow for air to pass through the anti acid G/tube pad. Using this type of material for the outer layer 50 would mean that the patient's gastric acid would be contained inside the anti acid G/tube pad.

The second layer 40 is an absorbent layer that is made of natural or synthetic material. This layer is designed to absorb the patient's gastric acid that is excreted from the G/tube site. The absorbent layer is typically constructed of a fibrous woven or nonwoven material. Woven gauze is a preferred material for the absorbent layer. Woven gauze has long been accepted as the standard material for constructing dressings, bandages and similar products.

The third layer 30 or the wicking layer is made of a wicking material. The wicking layer's purpose is to pull the gastric acid away from the patient's skin and allow it to pass through into the absorbent layer 40.

The fourth layer is the inner layer 20. The inner layer 20 is made of a permeable material that of which is similar to a feminine disposable napkin or a disposable diaper. It allows gastric acid to flow through it to the wicking layer. The inner layer 20 optionally but preferably has an adhesive edge 17 that is designed to help adhere the anti acid G/tube pad to the patient's skin and to keep the anti acid G/tube pad in position. There is a removable protective paper 21 that covers the adhesive edge 17 to protect the adhesive 18 until it is ready for use. The inner layer 20 will contain a dose of antacid, e.g., a dose of 1.3 grams of magnesium oxide and 1 gram of calcium carbonate, to help neutralize and prevent breakdown of the patient's tissue that surrounds the G/tube. The antacid, e.g., magnesium oxide and calcium carbonate, can be injected into or onto inner layer 20 or otherwise disposed thereon, or it may be in the form of a coating that is placed on the exposed surface of the inner layer 20 in the form of a dispersed layer on the inner layer 20. The antacid, e.g., magnesium oxide and calcium carbonate, 19 can be distributed in the inner layer 20 of the anti acid G/tube pad in a spiral fashion. The antacid, e.g., magnesium oxide and calcium carbonate, can be uniformly distributed throughout the layer. Other distributions, e.g., distributions as depicted in FIGS. 2A-2F of U.S. Pat. No. 6,599,525, are contemplated. The inner layer 20 can have an antacid free area 14 that is from 1 to 99, preferably 30 to 50 percent of the anti acid G/tube pad. This free area allows for the patient's gastric acids to pass through to the wicking layer 30.

The materials useful in constructing the layer or layers of the anti acid G/tube pad of this invention are known to those skilled in the art.

The permeable inner layer can be constructed of materials known per se in the art.

The wicking layer can be fabricated from materials known per se for this purpose.

The permeable or semi-permeable layer may comprise a porous polyethylene film, net or mesh. The layer may also be fabricated of polyvinyl chloride, polypropylene, polyester, nylon or the like.

It is preferred that the antacid composition be contained primarily or exclusively in or on the permeable inner layer. However, the antacid composition may be distributed in the absorbent layer and in or on the inner layer.

Only certain forms of this invention have been illustrated and described. It should be appreciated that the compositions and methods of the present invention are capable of being incorporated in the form of a variety of embodiments, only a few of which have been illustrated and described above. The invention may be embodied in other forms without departing from its spirit or essential characteristics. The described embodiments are to be considered in all respects only as illustrative and not restrictive, and the scope of the invention, therefore, indicated by the appended claims rather than by the foregoing description. All changes which come within the meaning and range of equivalency of the claims are to be embraced within their scope.

1. An anti acid G/Tube pad comprising:
a permeable layer adapted to face the skin, sad permeable layer having thereon an antacid active, and
an absorbent layer.

2. The pad of claim 1 further comprising a hole in the center thereof adapted to fit around a G-Tube.

3. The pad of claim 2 further comprising a slit from one end of said pad to said hole.

4. The pad of claim 1 further comprising a wicking layer disposed between said permeable layer and said absorbent layer.

5. The pad of claim 1 further comprising a semipermeable layer disposed on said absorbent layer.

6. The pad of claim 1 wherein said antacid active is disposed on said inner layer in a discontinuous manner such that 1-99% permeable layer remains exposed to the skin.

7. A method of treating a patient in need of a G-Tube comprising administering to said patient the anti acid G/Tube pad of claim 1.
